(19) United States
(12) Patent Application Publication
(10) Pub. No.: US 2026/0188096 A1
(43) Pub. Date: Jul. 2, 2026

(54) NON-FACE-TO-FACE NON-CONTACT FALL MONITORING SYSTEM, NON-CONTACT SLEEP MONITORING SYSTEM AND METHOD

(71) Applicant: JCF TECHNOLOGY INC., Seoul (KR)

(72) Inventor: Jin Myung KIM, Bucheon-si, Gyeonggi-do (KR)

(21) Appl. No.: 18/840,916

(22) PCT Filed: Apr. 10, 2024
(86) PCT No.: PCT/KR2024/004784

§ 371 Date: Aug. 23, 2024

(30) Foreign Application Priority Data
Apr. 11, 2023 (KR) 10-2023-0047850
Apr. 11, 2023 (KR) 10-2023-0047853

(51) Int. Cl. G08B 21/04; A61B 5/00; A61B 5/0205

(52) U.S. Cl. CPC G08B 21/043; A61B 5/0205; A61B 5/4812; G08B 21/0476; G08B 21/0492

(57) ABSTRACT

The present invention is implemented as a non-face-to-face non-contact fall monitoring system, and a non-contact sleep monitoring system and method, which improve reliability of fall detection through detection of a fall of a user using radar signals and thermal image signals, and analyze a sleep state of the user and provides guidance thereon.

### TECHNICAL FIELD

The present invention relates to a non-face-to-face non-contact fall monitoring system that improves reliability of fall detection through detection of a fall of a user using radar signals and thermal image signals, and a non-contact sleep monitoring system and method that analyzes a sleep state of the user and provides guidance thereon.

### BACKGROUND ART

Recently, the number of people in need of care or attention is increasing in the domestic society.

The people in need of care or attention may be defined as people who need assistance when an emergency situation occurs or people who do not have a housemate, and for example, the elderly, the disabled, and single-person households may be included. According to a recent survey, the elderly population in Korea has exceeded 9 million, and the population of single-person households and registered disabled people has also exceeded 6.6 million and 2.6 million, respectively. In addition, the number of people who die alone due to absence of a housemate exceeds 10,000.

Accordingly, falling accidents, which are accidents gradually increasing, are accidents in which a person falls regardless of his or her will, and are dangerous accidents that may develop various complications and even lead to death.

Therefore, fall detection techniques that can accurately detect falling accidents and quickly respond thereto are important.

Fall detection devices using cameras detect movement of an elderly person through video and alert the guardian when a fall is detected, but there is a problem of violating human rights and privacy issues.

In addition, fall detection devices using radar signals of the prior art have a problem in that a fall is not detected although the fall actually occurs or a fall is detected although a fall does not occur due to radio interference by wind or the like.

In addition, since wearable fall detection devices of the prior art have a disadvantage in that it is unable to properly analyze fall of a user since the user often forgets to wear the wearable devices or loses the wearable devices in many cases.

In addition, when such single-person households or the like lead to irregular lives, quality of sleep comes to be irregular, and this may impair health in some cases. In order to measure the quality of sleep, it needs to go through a troublesome process of making an appointment at a specialized hospital, visiting the hospital on time, and using specialized devices while sleeping, and therefore, there is a problem in that it is difficult to measure the quality of sleep regularly.

### DISCLOSURE OF INVENTION

### Technical Problem

The present invention has been made in view of the above problems, and it is an object of the present invention to provide a non-face-to-face non-contact fall monitoring system that can increase reliability of fall detection while protecting privacy of a user.

In addition, another object of the present invention is to provide a non-face-to-face non-contact fall monitoring system that can monitor fall of a user without wearing a detection device.

In addition, another object of the present invention is to provide a non-face-to-face non-contact fall monitoring system that determines a risk level when a fall occurs and provides a risk alert to guardians or medical staff.

In addition, another object of the present invention is to provide a non-contact sleep monitoring system and method that enhances reliability of analyzing a state of a user while the user sleeps, by acquiring biological activity information while the user is in a sleeping state using a radar sensor unit, and acquiring a tossing and turning motion as an auxiliary signal using a thermal imaging sensor unit.

In addition, another object of the present invention is to provide a non-contact sleep monitoring system and method that can protect privacy of a user as thermal image data is not directly used.

### Technical Solution

A non-face-to-face non-contact fall monitoring system of the present invention may comprise: a radar sensor unit for transmitting a radar signal toward a user, receiving the radar signal reflected from the user, and generating a time series Doppler radar signal using a Doppler radar algorithm; a thermal image sensor unit for generating thermal image data by photographing the user from a top of the user; a radar signal pattern analysis unit for receiving the time series Doppler radar signal from the radar sensor unit, and analyzing a state of the user on the basis of the received time series Doppler radar signal; a thermal image data processing unit for receiving the generated thermal image data from the thermal image sensor unit, and analyzing the state of the user by processing the received thermal image data; and a fall determination unit for determining whether or not a fall has occurred on the basis of state data of the user received from the radar signal pattern analysis unit and the thermal image data processing unit.

The radar signal pattern analysis unit may separate a movement change signal and a biological signal from the time series Doppler radar signal received from the radar sensor unit, and analyze the state of the user using the separated movement change signal as a time series movement Doppler radar signal.

The radar signal pattern analysis unit may generate a first state data indicating that a fall has occurred, when an amplitude size becomes 0 after a yin-yang change occurs in a data having an amplitude size of a predetermined level or higher in the time series movement Doppler radar signal within a first time, and generate a second state data indicating a state in which the user slowly falls down and a fall is suspected, when a yin-yang change occurs in the data having an amplitude size of a predetermined level or higher in the time series movement Doppler radar signal after the first time.

The thermal image data processing unit may process the thermal image data through the steps of: determining an analysis range from the received thermal image data; determining an edge of the user within the determined analysis range; and acquiring a closed-curved image by processing the thermal image data to have a closed-curved shape from the determined edge using a preset threshold value.

The thermal image data processing unit may form a labeled image by further including the step of performing image labeling on the closed-curved image.

The thermal image data processing unit may form a spectrogram by further including the step of forming a spectrogram of the labeled image.

The thermal image data processing unit may form fourth to sixth state data by determining the state of the user as one among fourth to sixth states based on at least one among the closed-curved image, the labeled image, and the spectrogram.

The fourth state data may indicate a state in which the user is positioned on an object at a predetermined height from a floor, the fifth state data may indicate a state in which the user is falling from the object positioned at a predetermined height from the floor, and the sixth state data may indicate a state in which the user has fallen from the object positioned at a predetermined height from the floor.

When the fall determination unit receives the first state data from the radar signal pattern analysis unit, and sequentially receives all the fourth state data, the fifth state data, and the sixth state data from the thermal image data processing unit, and the fourth state data is generated before a time point of generating the first state data, the fall determination unit may determine that the user has fallen, and generate a first signal indicating that a fall of the user has occurred; when the fall determination unit receives the first state data determined as a first state from the radar signal pattern analysis unit, and sequentially receives all the sixth state data, the fifth state data, and the fourth state data from the thermal image data processing unit, and the sixth state data is generated before the time point of generating the first state data, the fall determination unit may determine that the user has not fallen, and generate a second signal indicating that a fall has not occurred; when the fall determination unit receives the second state data indicating that a fall is suspected from the radar signal pattern analysis unit, and sequentially receives all the fourth state data, the fifth state data, and the sixth state data from the thermal image data processing unit, and the fourth state data is generated before the time point of generating the first state data, the fall determination unit may determine that the user has fallen, and generate a third signal indicating that the user slowly falls down and a fall of the user has occurred; when the fall determination unit receives the third state data from the radar signal pattern analysis unit, and receives only the fourth state data from the thermal image data processing unit even after a fourth time from a time point of receiving the third state data, the fall determination unit may generate a fourth signal indicating that the user is at a high risk of falling; and when the fall determination unit receives the first state data or the second state data from the radar signal pattern analysis unit, sequentially receives all the fourth state data, the fifth state data, and the sixth state data from the thermal image data processing unit, receives again the first state data or the second state data from the radar signal pattern analysis unit after the fourth time, and sequentially receives the sixth state data, the fifth state data, and the fourth state data from the thermal image data processing unit, the fall determination unit may determine that the fallen user has returned to an original position, and generate a fifth signal indicating that the user has returned to the original position.

The radar signal pattern analysis unit may analyze the separated biological signal, and generate the sixth signal informing a dangerous situation when the biological signal is different from a biological signal pattern of a predetermined period as much as a predetermined range or more.

The non-contact fall and sleep monitoring system may further comprise a fall risk level determination unit.

When the fall risk level determination unit continuously receives the sixth signal from the radar signal pattern analysis unit during a fifth time, receives the first signal from the fall determination unit, and does not continuously receive the fourth signal within the fifth time, the fall risk level determination unit may determine that the user is continuously in a fallen state and the biological signals are in a bad state, and generate a seventh signal informing that the user is in a very high risk due to the falling; when the fall risk level determination unit does not receive the sixth signal from the radar signal pattern analysis unit during the fifth time, and does not receive the fourth signal from the fall determination unit during the fifth time, the fall risk level determination unit may generate an eighth signal indicating that the biological signals of the user are stable although the user is continuously in a fallen state; and when the fall risk level determination unit receives the third signal from the fall determination unit, the fall risk level determination unit may generate a ninth signal informing that it is high probable that the user may fall.

The non-face-to-face non-contact fall monitoring system may further comprise an alarm unit, wherein when the seventh signal is received from the fall risk level determination unit, the alarm unit may generate a first alarm signal informing that the user is in a very critical state, and transmit the first alarm signal to both a predetermined guardian and medical staff; when the eighth signal is received from the fall risk level determination unit, the alarm unit may generate a second alarm signal informing a fallen state of the user, and transmit the second alarm signal to a predetermined guardian or medical staff; and when the ninth signal is received from the fall risk level determination unit, the alarm unit may generate a third alarm signal informing that falling of the user is imminent, and transmit the third alarm signal to a predesignated guardian or medical staff.

A non-contact sleep monitoring system and method of the present invention may comprise: a Doppler signal acquisition unit for acquiring a Doppler signal including biological activity information using a radar; an auxiliary signal processing unit for acquiring a tossing and turning motion as an auxiliary signal using a thermal image sensor; a Doppler signal analysis unit for acquiring spectrum energy at preset intervals by analyzing the Doppler signal, determining whether the spectrum energy is acquired periodically, and performing, when the spectrum energy is determined as non-periodic spectrum energy, classification of the non-periodic spectrum energy using the auxiliary signal; and a sleep-stage definition unit for defining a sleep state of each stage in an entire sleep stage using a combination of a ratio of respiration spectrum energy and heartbeat spectrum energy, among the spectrum energy, and the non-periodic spectrum energy.

The Doppler signal may include a respiration Doppler signal that acquires information on breathing of a user and a heartbeat Doppler signal that acquires information on heartbeat of the user.

The Doppler signal analysis unit may acquire the spectrum energy by performing a fast Fourier transform on the Doppler signal.

The sleep-stage definition unit may define a stage in which the non-periodic spectrum energy does not exist, among stages where the ratio of respiration spectrum energy and heartbeat spectrum energy is 5:5 or higher in the entire sleep stage, as a deep sleep stage, and define a stage in which the non-periodic spectrum energy does not exist, among stages where the ratio of respiration spectrum energy and heartbeat spectrum energy is lower than 5:5, as an apnea stage.

The sleep-stage definition unit may define a stage in which the non-periodic spectrum energy exists and the non-periodic spectrum energy appears to be higher than or equal to a preset magnitude as a tossing and turning stage, and define a stage in which the non-periodic spectrum energy appears to be lower than or equal to a preset magnitude in the apnea stage as a snoring stage.

A non-contact sleep monitoring system and method according to another embodiment of the present invention may comprise: a Doppler signal acquisition unit for acquiring a Doppler signal including biological activity information using a radar; an auxiliary signal processing unit for acquiring a tossing and turning motion as an auxiliary signal using a thermal image sensor; a Doppler signal analysis unit for acquiring spectrum energy at preset intervals by analyzing the Doppler signal, determining whether the spectrum energy is acquired periodically, and performing classification of the spectrum energy using the auxiliary signal; and a sleep-stage definition unit for defining sleep states of the other sleep stages using a preset ratio range and the non-periodic spectrum energy on the basis of an average of the spectrum energy of a sleep entry stage that satisfies preset criteria in an entire sleep stage.

A non-contact sleep monitoring system and method of the present invention may comprise the steps of: a Doppler signal acquisition step of acquiring, by a Doppler signal acquisition unit, a Doppler signal including biological activity information using a radar; an auxiliary signal processing step of acquiring, by an auxiliary signal processing unit, a tossing and turning motion as an auxiliary signal using a thermal image sensor; a Doppler signal analysis step of acquiring, by a Doppler signal analysis unit, spectrum energy at preset intervals by analyzing the Doppler signal, determining whether the spectrum energy is acquired periodically, and performing classification of the spectrum energy using the auxiliary signal; and a sleep-stage definition step of defining, by a sleep-stage definition unit, a sleep state of each stage in an entire sleep stage using a combination of a ratio of respiration spectrum energy and heartbeat spectrum energy, among the spectrum energy, and the non-periodic spectrum energy.

The Doppler signal may include a respiration Doppler signal that acquires information on breathing of a user and a heartbeat Doppler signal that acquires information on heartbeat of the user.

The Doppler signal analysis step may acquire the spectrum energy by performing a fast Fourier transform on the Doppler signal.

The sleep-stage definition step may define a stage in which the non-periodic spectrum energy does not exist, among stages where the ratio of respiration spectrum energy and heartbeat spectrum energy is 5:5 or higher in the entire sleep stage, as a deep sleep stage, and define a stage in which the non-periodic spectrum energy does not exist, among stages where the ratio of respiration spectrum energy and heartbeat spectrum energy is lower than 5:5, as an apnea stage.

The sleep-stage definition step may define a stage in which the non-periodic spectrum energy exists and the non-periodic spectrum energy appears to be higher than or equal to a preset magnitude as a tossing and turning stage, and define a stage in which the non-periodic spectrum energy appears to be lower than or equal to a preset magnitude in the apnea stage as a snoring stage.

According to another aspect of the present invention, there is provided a non-contact sleep monitoring system and method, and the method may comprise the steps of: acquiring, by a Doppler signal acquisition unit, a Doppler signal including biological activity information using a radar; acquiring, by an auxiliary signal processing unit, a tossing and turning motion as an auxiliary signal using a thermal image sensor; acquiring, by a Doppler signal analysis unit, spectrum energy at preset intervals by analyzing the Doppler signal, determining whether the spectrum energy is acquired periodically, and performing classification of the spectrum energy using the auxiliary signal; and defining, by a sleep-stage definition unit, sleep states of the other sleep stages using a preset ratio range and the non-periodic spectrum energy on the basis of an average of the spectrum energy of a sleep entry stage that satisfies preset criteria in an entire sleep stage.

### Advantageous Effects

The present invention has an advantage of increasing reliability of fall detection while protecting privacy of a user.

In addition, the present invention has an advantage of determining fall of a user in real time in a non-face-to-face non-contact method, rather than a wearable method, although the user does not wear a separate device.

In addition, the present invention has an advantage of determining a risk level when it is determined as a fall and informing a guardian or medical staff of the risk so that the risk according to the fall can be prevented or responded in advance.

In addition, the present invention has an advantage of protecting privacy of a user by processing thermal images during fall monitoring to create a closed curve, a labeling image, and a spectrogram.

In addition, the present invention has an advantage of analyzing a risk level of a user and properly responding thereto on the basis of biological signals of the user and image-processed thermal images.

In addition, the present invention has an advantage of not disturbing user's sleep as biological activity information is acquired using a radar while the user sleeps without affecting the user's body.

In addition, the present invention has an advantage of accurately determining a sleep state in each sleep stage of a user using the user's breathing and heartbeat information.

In addition, the present invention has an advantage of improving reliability compared to existing Doppler signal-based sleep monitoring as sleep monitoring is conducted using the contour of a thermal image, together with a Doppler signal.

### BRIEF DESCRIPTION OF THE DRAWINGS

FIG. 1 is a view showing a non-face-to-face non-contact fall monitoring system according to an embodiment of the present invention.

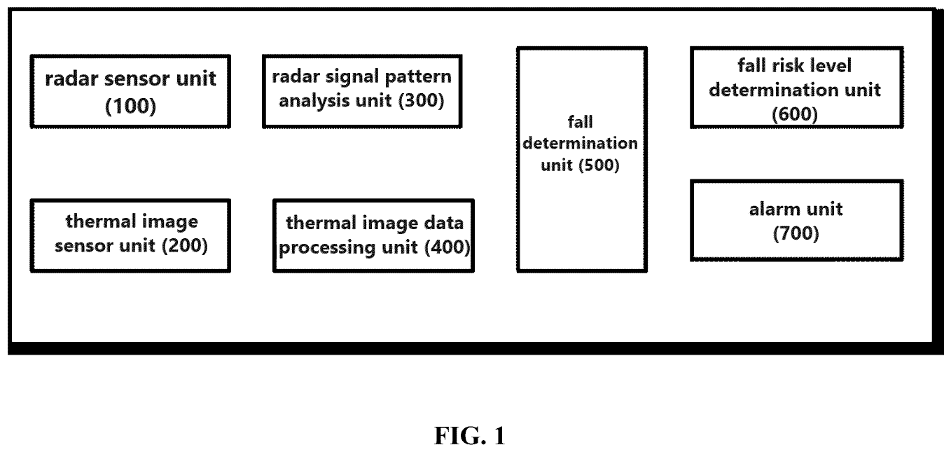

FIG. 2 is a view showing a movement pattern analyzed by a radar signal pattern analysis unit according to an embodiment of the present invention.

FIGS. 3A-3F are views showing a process of analyzing thermal image data and determining as a fourth state.

FIGS. 4A-4F are views showing a process of analyzing thermal image data and determining as a fifth state.

FIGS. 5A-5F are views showing a process of analyzing thermal image data and determining as a sixth state.

FIG. 6 is a block diagram showing a non-contact sleep monitoring system according to an embodiment of the present invention.

FIG. 7 is a flowchart illustrating a non-contact sleep monitoring method according to an embodiment of the present invention.

FIG. 8 is a graph showing the result of an actual simulation using an embodiment of the present invention.

FIGS. 9A-9F are views showing a process of processing thermal image data by an auxiliary signal processing unit of the present invention.

### BEST MODE FOR CARRYING OUT THE INVENTION

A non-face-to-face non-contact fall monitoring system of the present invention comprises: a radar sensor unit for transmitting a radar signal toward a user, receiving the radar signal reflected from the user, and generating a time series Doppler radar signal using a Doppler radar algorithm; a thermal image sensor unit for generating thermal image data by photographing the user from a top of the user; a radar signal pattern analysis unit for receiving the time series Doppler radar signal from the radar sensor unit, and analyzing a state of the user on the basis of the received time series Doppler radar signal; a thermal image data processing unit for receiving the generated thermal image data from the thermal image sensor unit, and analyzing the state of the user by processing the received thermal image data; and a fall determination unit for determining whether or not a fall has occurred on the basis of state data of the user received from the radar signal pattern analysis unit and the thermal image data processing unit.

### MODE FOR CARRYING OUT THE INVENTION

A non-face-to-face non-contact fall monitoring system according to an embodiment of the present invention may include a radar sensor unit (100), a thermal image sensor unit (200), a radar signal pattern analysis unit (300), a thermal image data processing unit (400), a fall determination unit (500), a fall risk level determination unit (600), and an alarm unit (700).

The non-face-to-face non-contact fall monitoring system according to an embodiment of the present invention may be installed a space where a user lives, as well as a space where the user sleeps.

### 1. Radar Sensor Unit (100)

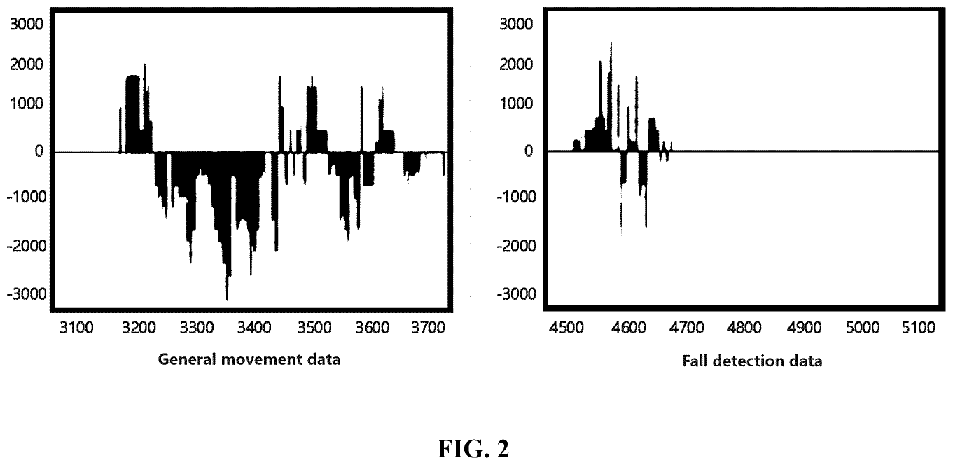

The radar sensor unit (100) may transmit a radar signal toward a user, receive the radar signal reflected from the user, and form a time series Doppler radar signal as shown in FIG. 2 using a Doppler radar algorithm. Here, the time series Doppler radar signal means that the X-axis represents the time and the Y-axis represents the magnitude of the Doppler radar signal. In the time series Doppler radar signal, directions of positive data and negative data on the Y-axis are opposite to each other, and the amplitude size, which is the absolute value on the Y-axis, represents the speed of movement of the user or the movement of a body part with a large area.

### 2. Thermal Image Sensor Unit (200)

The thermal image sensor unit (200) may be an infrared thermal imaging camera. The infrared thermal imaging camera may photograph a user from the top of the user. Thermal image data of the photographed user is transmitted to a determination unit. Preferably, the infrared thermal imaging camera may be installed on the ceiling above the bed in the bedroom.

### 3. Radar Signal Pattern Analysis Unit (300)

The radar signal pattern analysis unit (300) may separate a movement change signal and a biological signal from the time series Doppler radar signal received from the radar sensor unit (100). Hereinafter, the movement change signal separated from the time series Doppler radar signal will be referred to as a time series movement Doppler radar signal.

When the time series movement Doppler radar signal is analyzed, directions of the Y-axis positive phase and negative phase are opposite to each other. Therefore, when phase change occurs on the Y-axis from the positive phase to the negative phase, it means that the direction of the second movement has changed to the opposite direction of the first movement, such as moving from front to back, from back to front, from top to bottom, or from bottom to top. Referring to FIG. 2, Doppler time series data corresponding to a fall shows a change in the phase in order of positive, negative, positive, negative, and positive, which means that movement of the entire body or a body part of a user has changed in the opposite directions four times in total.

In addition, the amplitude size (absolute value of the Y-axis) of the time series movement Doppler radar signal means the speed or movement size or both of them. That is, when the amplitude is large, it means that the movement is fast or the area of movement is large, like the movement of the arms or legs, rather than the biological signal.

Therefore, when the direction of a body part with a large area, such as an arm or a leg, changes rapidly, the phase of a large amplitude in the Y-axis may change from plus to minus or from minus to plus. In the present invention, the change in the Y-axis phase from plus to minus or from minus to plus is called a yin-yang change.

The radar signal pattern analysis unit (300) generates a first state data indicating that a fall has occurred, when the amplitude size becomes 0 after a yin-yang change occurs in the data having an amplitude size of a predetermined level or higher in the time series movement Doppler radar signal within a first time.

As an example, the radar signal pattern analysis unit (300) generates a first state data indicating that a fall has occurred when the amplitude size is greater than or equal to a predetermined range and the amplitude of the time series movement Doppler radar signal is constant at 0 after a yin-yang change is generated within 200 ms.

In addition, the radar signal pattern analysis unit (300) may generate a second state data indicating a state in which the user slowly falls down and a fall is suspected, when a yin-yang change occurs in the data having an amplitude size of a predetermined level or higher in the time series movement Doppler radar signal after the first time.

In addition, the radar signal pattern analysis unit (300) may generate a third state data indicating that the user continuously moves although not falling, when the amplitude size of the time series movement Doppler radar signal is greater than a predetermined range based on the analysis of the time series movement Doppler radar signal, and the amplitude of a second time series movement Doppler radar signal is generated after a second time after a first time series movement Doppler radar signal is generated.

When the second state data is generated while the time series movement Doppler radar signal is analyzed, there is a disadvantage in that whether the user had fallen may not be accurately determined.

In addition, as the change in the direction of limbs occurs quickly when the user jumps from under the bed onto the bed contrary to the fall, the time series movement Doppler radar signal analysis sometimes misdiagnoses the jump as a fall since the amplitude of the movement Doppler radar signal is large and a yin-yang change occurs within a predetermined time period.

In addition, the time series movement Doppler radar signal analysis has a disadvantage in that when a person is lying at the edge of a bed and highly likely to fall although a fall has not been occurred actually, as it is unable to determine the situation as a fall and determines a fall only after a fall actually occurs, it may not prevent the fall.

To complement these disadvantages, the present invention uses thermal image data analysis, together with time series movement Doppler radar signal analysis.

### 4. Thermal Image Data Processing Unit (400)

The thermal image data processing unit (400) may analyze a state of the user by processing the thermal image data received from the thermal image sensor unit (200).

The thermal image data processing unit (400) determines an analysis range from the received thermal image data. Thereafter, the thermal image data processing unit (400) determines the edge of the user within the determined analysis range. Thereafter, the thermal image data processing unit (400) may acquire a closed-curved image by processing the thermal image data to have a closed-curved shape from the determined edge using a preset threshold value.

Thereafter, the thermal image data processing unit (400) may form a labeled image by further performing image labeling on the closed-curved image. Thereafter, the thermal image data processing unit (400) may also form a spectrogram of the labeled image.

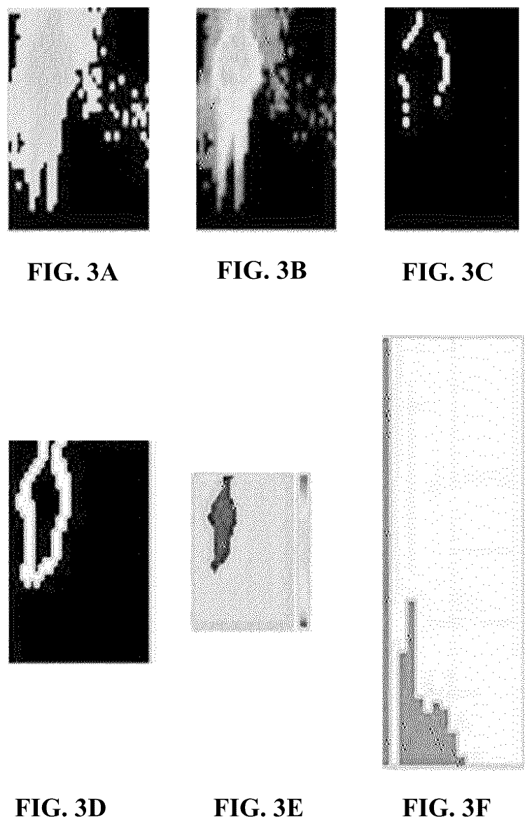

FIGS. 3A-3F are views showing a process of analyzing the thermal image data received from the thermal image sensor unit (200) and determining as a fourth state. Here, the fourth state means a state in which the user is positioned on an object at a predetermined height from the floor (e.g., lying on the bed). FIG. 3A shows thermal image data received from the thermal image sensor unit (200), FIG. 3B shows thermal image data of which the analysis range has been determined, FIG. 3C shows the edge of the user, FIG. 3D shows a closed-curved image of the user, FIG. 3E shows a labeled image of the user, and FIG. 3F shows a spectrogram of the labeled image of the user.

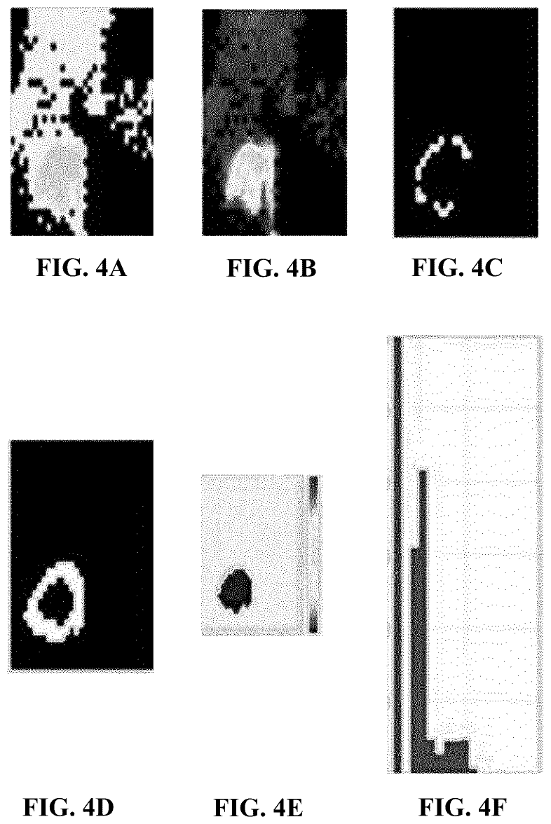

FIGS. 4A-4F are views showing a process of analyzing the thermal image data received from the thermal image sensor unit (200) and determining as a fifth state. Here, the fifth state means a state in which the user is falling from an object (e.g., a bed) positioned at a predetermined height from the floor. FIG. 4A shows thermal image data received from the thermal image sensor unit (200), FIG. 4B shows thermal image data of which the analysis range has been determined, FIG. 4C shows the edge of the user, FIG. 4D shows a closed-curved image of the user, FIG. 4E shows a labeled image of the user, and FIG. 4F shows a spectrogram of the labeled image of the user.

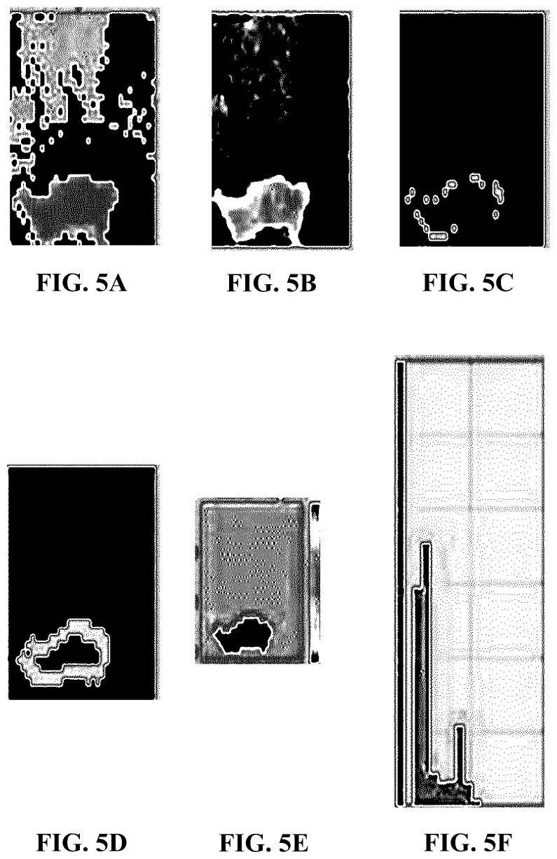

FIGS. 5A-5F are views showing a process of analyzing the thermal image data received from the thermal image data processing unit (400) and determining as a sixth state. Here, the sixth state means a state in which the user has fallen from an object (e.g., a bed) positioned at a predetermined height from the floor. FIG. 5A shows thermal image data received from the thermal image sensor unit (200), FIG. 5B shows thermal image data of which the analysis range has been determined, FIG. 5C shows the edge of the user, FIG. 5D shows a closed-curved image of the user, FIG. 5E shows a labeled image of the user, and FIG. 5F shows a spectrogram of the labeled image of the user.

The thermal image data processing unit (400) may separately determine the fourth state in which the user is lying on an object (e.g., a bed) of a predetermined height, the fifth state in which the user is falling from an object (e.g., a bed) of a predetermined height, and the sixth state in which the user has fallen from an object (e.g., a bed) of a predetermined height, on the basis of at least one among the closed-curved images (3d, 4d, and 5d), the labeled images (3e, 4e, and 5e), and the spectrograms (3f, 4f, and 5f), generate fourth state data indicating the fourth state, fifth state data indicating the fifth state, and sixth state data indicating the sixth state, and transmit the fourth to sixth state data generated in this way to the fall determination unit (500), together with the time points of generating the fourth to sixth state data.

Preferably, the thermal image data processing unit (400) may determine the fourth to sixth states through analysis of the shape of the spectrogram, generate fourth state data indicating the fourth state, fifth state data indicating the fifth state, and sixth state data indicating the sixth state, and transmit the fourth to sixth state data generated in this way to the fall determination unit (500), together with the time points of generating the fourth to sixth state data.

### 5. Fall Determination Unit (500)

The fall determination unit (500) may determine whether or not a fall has occurred and analyze a fall risk level by matching the first to third state data received from the radar signal pattern analysis unit (300) and the fourth to sixth state data received from the thermal image data processing unit (400) at the same time point, and matching at least one among the first to third state data and the fourth to sixth state data.

### (1) Case Determined as a Fall (Generation of First Signal)

When the fall determination unit (500) receives the first state data determined as the first state from the radar signal pattern analysis unit (300), and sequentially receives all the fourth state data, the fifth state data, and the sixth state data from the thermal image data processing unit (400), and the fourth state data is generated before the time point of generating the first state data, the fall determination unit (500) may determine that the user has fallen, generate a first signal indicating that a fall of the user has occurred, and transmit the first signal to the fall risk level determination unit (600).

### (2) Case Determined as not a Fall (Generation of Second Signal)

On the contrary, when the fall determination unit receives the first state data determined as the first state from the radar signal pattern analysis unit (300), and sequentially receives all the sixth state data, the fifth state data, and the fourth state data from the thermal image data processing unit (400), and the sixth state data is generated before the time point of generating the first state data, the fall determination unit may determine that the user has not fallen, but jumped onto an object located above a predetermined height, generate a second signal indicating that a fall has not occurred, and transmit the second signal to the fall risk level determination unit (600).

### (3) Determine as a Fall Occurred at a Slow Speed (Generation of Third Signal)

When the fall determination unit (500) receives the second state data indicating that a fall is suspected from the radar signal pattern analysis unit (300), and sequentially receives all the fourth state data, the fifth state data, and the sixth state data from the thermal image data processing unit (400), and the fourth state data is generated before the time point of generating the first state data, the fall determination unit may determine that the user has fallen, generate a third signal indicating that the user slowly falls down and a fall of the user has occurred, and transmit the third signal to the fall risk level determination unit (600).

(4) Determine that Fall Risk Level is High (Generation of Fourth Signal)

When the fall determination unit (500) receives the third state data determined as the third state from the radar signal pattern analysis unit (300), and receives only the fourth state data from the thermal image data processing unit (400) even after a third time from the time point of receiving the third state data, the fall determination unit may determine that the user is at a high risk of falling, generate a fourth signal indicating a high risk of falling, and transmit the fourth signal to the fall risk level determination unit (600).

(5) Determined to have Returned to the Original Position (Generation of Fifth Signal)

When the fall determination unit (500) receives the first state data determined as the first state from the radar signal pattern analysis unit (300), sequentially receives all the fourth state data, the fifth state data, and the sixth state data from the thermal image data processing unit (400), receives again the first state data determined as the first state from the radar signal pattern analysis unit (300) after a fourth time, and sequentially receives the sixth state data, the fifth state data, and the fourth state data from the thermal image data processing unit (400), the fall determination unit may determine that the fallen user has returned to the original position, generate a fifth signal indicating that the user has returned to the original position, and transmit the fifth signal to the fall risk level determination unit (600).

### (5) Bad Biological Signal (Generation of Sixth Signal)

The radar signal pattern analysis unit (300) may analyze the separated biological signal, generate a sixth signal informing a dangerous situation when the biological signal is different from a biological signal pattern of a predetermined period as much as a predetermined range or more, and transmits the sixth signal to the fall risk level determination unit (600). For example, when the heart rate or respiration rate, which are biological signals, decrease or increase as much as 50% or more compared to the average heart rate or respiration rate for one month or one week, the radar signal pattern analysis unit (300) may generate a sixth signal informing that the user is in a dangerous state as the biological signals of the user are bad.

### 6. Fall Risk Level Determination Unit (600)

The fall risk level determination unit (600) may receive the sixth signal from the radar signal pattern analysis unit (300) and the first to fifth signals from the fall determination unit (500), separately analyze the risk level according to fall of a user, and generate a signal indicating each risk.

### (1) Risk Level of Falling is Very High (Generation of Seventh Signal)

When the fall risk level determination unit (600) continuously receives the sixth signal from the radar signal pattern analysis unit (300) during a fifth time, receives the first signal from the fall determination unit (500), and does not continuously receive the fourth signal within the fifth time, the fall risk level determination unit may determine that the user is continuously in a fallen state (i.e., unable to get on the bed due to the fall) and the biological signals are continuously in a bad state for a predetermined period of time, generate a seventh signal informing that the user is in a very high risk due to the falling, and transmit the seventh signal to the alarm unit (700).

### (2) Risk Level of Falling is Very High (Generation of Seventh Signal)

When the fall risk level determination unit (600) continuously receives the sixth signal from the radar signal pattern analysis unit (300) during a fifth time, receives the third signal from the fall determination unit (500), and does not continuously receive the fourth signal within the third time, the fall risk level determination unit may determine that the user is continuously in a fallen state (i.e., unable to get on the bed due to the fall) and the biological signals are continuously in a bad state for a predetermined period of time, generate a seventh signal informing that the user is in a very high risk due to the falling, and transmit the seventh signal to the alarm unit (700).

### (3) Fallen State Continues, but Biological Signals are Stable (Eighth Signal Generated)

When the fall risk level determination unit (600) does not receive the sixth signal from the radar signal pattern analysis unit (300) during the fifth time, receives the third signal from the fall determination unit (500), and does not continuously receive the fourth signal within the third time, since the biological signals are not bad although the user is continuously in a fallen state (i.e., unable to get onto the bed due to the fall), the fall risk level determination unit may generate an eighth signal informing that the user is not in a high risk due to the falling and transmit the eighth signal to the alarm unit (700).

### (4) Highly Probable to Fall (Generation of Ninth Signal)

When the fall risk level determination unit (600) receives the fourth signal from the fall determination unit (500), it may generate a ninth signal informing that it is high probable that the user may fall, and transmit the ninth signal to the alarm unit (700).

### (4) No Fall has Occurred (Generation of No Signal)

When the fall risk level determination unit (600) receives the second signal from the fall determination unit (500), it analyzes that the risk level of the fallen state of the user is low and does not separately generate a signal.

### (5) Fallen State is Finished and Biological Signal is Stabilized (Generation of No Signal)

When the fall risk level determination unit (600) does not receive the sixth signal from the radar signal pattern analysis unit (300) within the fifth time, and receives the fourth signal within the fifth time, it determines that there is no risk to the safety of the user and does not generate a separate signal as the biological signals of the user are stable and the fallen state of the user is finished (i.e., the user has climbed onto the bed on his own).

### 7. Alarm Unit (700)

When the seventh signal is received from the fall risk level determination unit (600), the alarm unit (700) may generate a first alarm signal informing that the user is in a fallen and very critical state in which the biological signals are bad, and transmit the first alarm signal to both a predetermined guardian and medical staff.

When the eighth signal is received from the fall risk level determination unit (600), the alarm unit (700) may generate a second alarm signal informing that the user is in a fallen state but the biological signals are stable, and transmit the second alarm signal to a predetermined guardian or medical staff.

When the ninth signal is received from the fall risk level determination unit (600), the alarm unit (700) may generate a third alarm signal informing that falling of the user is imminent, and transmit the third alarm signal to a predesignated guardian or medical staff.

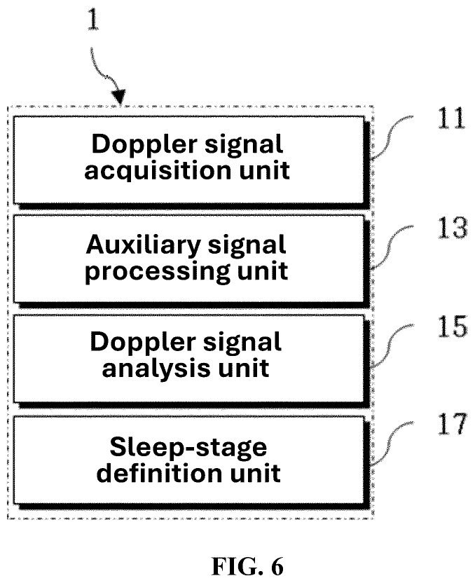

FIG. 6 is a block diagram showing a non-contact sleep monitoring system according to an embodiment of the present invention. A non-contact sleep monitoring system (1) according to an embodiment of the present invention may be formed to be installed in a space where a user lives and sleeps, such as a master bedroom or the like, to detect sleep when the user falls asleep, acquire Doppler signals for biological activities, and define a sleep state of the user by analyzing the acquired Doppler signals. To this end, the present invention may use a biological activity measurement device using a radar previously installed in the space described above. As shown in FIG. 6, the non-contact sleep monitoring system (1) according to an embodiment of the present invention may be formed to include a Doppler signal acquisition unit (11), an auxiliary signal processing unit (13), a Doppler signal analysis unit (15), and a sleep-stage definition unit (17).

The Doppler signal acquisition unit (11) is formed to acquire a Doppler signal including biological activity information from a radar installed in a user's sleeping space. The Doppler signal is formed to include a respiration Doppler signal, which is information on breathing of the user, and a heartbeat Doppler signal, which is information on heartbeat of the user. The Doppler signal may be defined as a signal that includes a Doppler frequency generated by movement of the user when the radar transmits radio waves to the user and the transmitted radio waves return. In an embodiment of the present invention, the respiration Doppler signal and the heartbeat Doppler signal may be Doppler signals with respect to the change in the movement of body organs that move while breathing and body organs that move while the heart is beating.

The auxiliary signal processing unit (13) is formed to acquire an auxiliary signal using an auxiliary biological activity information acquisition device further provided in the user's sleeping space, and acquire information on the movement (tossing and turning, or the like) of body parts, other than breathing or heartbeat, of the user, or the like by analyzing the acquired auxiliary signal. To this end, the auxiliary signal processing unit (13) may acquire measurement results from a thermal image sensor, which is an auxiliary biological activity information acquisition device. The thermal image sensor may be provided to measure movement of the body of the user.

The auxiliary signal processing unit (13) may continuously acquire contour information of the user's body using the thermal image sensor. When contour information of the user's body is continuously acquired using the thermal image sensor, changes in the contour information generated due to the movement, such as tossing and turning or the like, of the user can be acquired.

Preferably, the auxiliary signal processing unit (13) may continuously acquire information on the movement of the user by analyzing the thermal image data acquired from the thermal image sensor. In the present invention, information on the movement of the user may be acquired through the contour information or spectrogram, rather than using thermal image data as is, to protect privacy of the user.

The auxiliary signal processing unit (13) may analyze movement of the user by processing the thermal image data received from the thermal image sensor.

The auxiliary signal processing unit (13) determines an analysis range from the received thermal image data. Thereafter, the auxiliary signal processing unit (13) determines the edge of the user within the determined analysis range. Thereafter, the auxiliary signal processing unit (13) may acquire a closed-curved image by processing the thermal image data to have a closed-curved shape from the determined edge using a preset threshold value.

Thereafter, the auxiliary signal processing unit (13) may form a labeled image by further performing image labeling on the closed-curved image. Thereafter, the auxiliary signal processing unit (13) may also form a spectrogram of the labeled image.

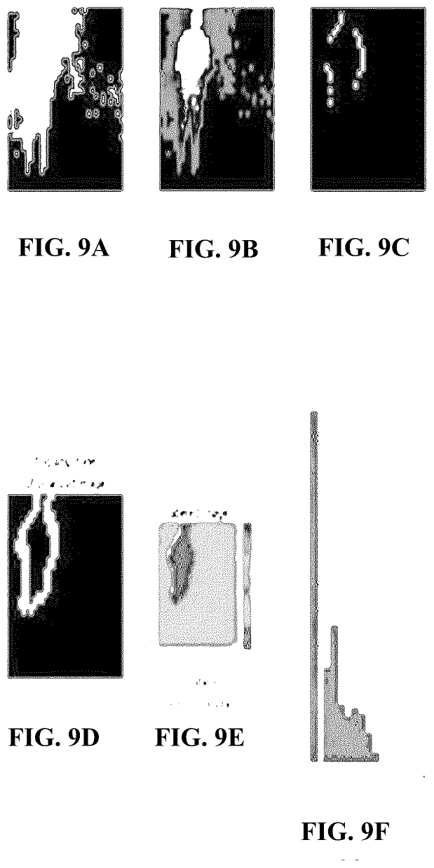

FIGS. 9A-9F are views showing a process of acquiring information on the movement of the user in the auxiliary signal processing unit (13). FIG. 9A shows thermal image data acquired from the thermal image sensor, FIG. 9B shows thermal image data of which the analysis range has been determined, FIG. 9C shows the edge of the user, FIG. 9D shows a closed-curved image of the user, FIG. 9E shows a labeled image of the user, and FIG. 9F shows a spectrogram of the labeled image of the user.

The body contour information acquired by the auxiliary signal processing unit (13) may be used by the Doppler signal analysis unit (15) described below, and as an example, when the Doppler signal analysis unit (15) acquires non-periodic spectrum energy using information on the time of acquiring a tossing and turning motion expressed as non-periodic spectrum energy, the non-periodic spectrum energy may be used as auxiliary information for determining what kind of biological activity information the energy means.

The Doppler signal analysis unit (15) is formed to acquire spectrum energy at preset intervals by analyzing the Doppler signals acquired through the Doppler signal acquisition unit (11). The Doppler signal analysis unit (15) may acquire spectrum energy by applying a preset function to the Doppler signals, and acquire respiration spectrum energy and heartbeat spectrum energy, which are spectrum energies of the respiration Doppler signal and the heartbeat Doppler signal, by analyzing the acquired spectrum energy according to preset criteria.

In an embodiment of the present invention, the Doppler signal analysis unit (15) may use a fast Fourier transform (FFT) as the preset function used to acquire spectrum energy from the Doppler signals at preset intervals. The Fourier transform is a well-known function and means a transformation that decomposes signals sampled in time or space into components of time frequency or space frequency. When the Fourier transform is used, the Doppler signal analysis unit (15) may convert the Doppler signals acquired at preset intervals into spectrum energy of a specific frequency component.

The Doppler signal analysis unit (15) according to an embodiment of the present invention is formed to confirm, when the spectrum energy of a specific frequency component is acquired, whether the acquired spectrum energy of a specific frequency component is spectrum energy generated periodically. The Doppler signal analysis unit (15) may be formed to continuously acquire spectrum energy of the Doppler signals acquired at preset intervals and determine whether the acquired continuous spectrum energy has periodicity. Here, when the spectrum energy has periodicity, the spectrum energy may be determined as spectrum energy of breathing or heartbeat, and when the spectrum energy has no periodicity, the spectrum energy may not be determined as spectrum energy of breathing or heartbeat.

The Doppler signal analysis unit (15) may confirm whether the non-periodic spectrum energy has an energy higher than a preset magnitude, and when it has an energy higher than a preset magnitude, the spectrum energy may be determined as spectrum energy of the Doppler frequency generated due to body movement of the user. In addition, it may be formed to determine, when the spectrum energy has an energy lower than a preset magnitude, the spectrum energy as spectrum energy of the Doppler frequency generated due to snoring of the user.

In addition, the Doppler signal analysis unit (15) according to an embodiment of the present invention may perform analysis on periodic spectrum energy using a preset determination algorithm, and separately acquire respiration spectrum energy and heartbeat spectrum energy as a result of the analysis.

In addition, the Doppler signal analysis unit (15) according to an embodiment of the present invention may identify the type of the non-periodic spectrum energy using the auxiliary signal acquired by the auxiliary signal processing unit (13) described above. Here, the Doppler signal analysis unit (15) may be formed to acquire non-periodic spectrum energy of tossing and turning (body movement) of the user using, for example, thermal image information, and determine the acquired non-periodic spectrum energy as a noise signal and remove it from data for analyzing sleep stages described below.

In addition, in another embodiment, when an energy of a preset magnitude that can distinguish body movement from snoring using the non-periodic spectrum energy is not defined, the Doppler signal analysis unit (15) may define an energy of a preset magnitude that can distinguish the two movements using the auxiliary signal acquired through a thermal image sensor.

The sleep-stage definition unit (17) is formed to define, when the Doppler signal analysis unit 15 according to an embodiment of the present invention continuously acquire spectrum energy of a specific frequency component, and determining whether or not the spectrum energy periodically occurs is completed, a sleep state in each stage of the entire sleep stage using an analysis result.

The entire sleep stage may include at least one among a sleep entry stage, a deep sleep stage, a tossing and turning stage, an apnea stage, and a snoring stage. The sleep entry stage means a stage in which the user enters sleep, and this is a stage in which whether the user begins to sleep can be determined, which occurs statistically in almost all users. In addition, the deep sleep stage means a sleep stage in which physical activities of the user are maintained stably, while the tossing and turning stage means a sleep stage in which the physical activities of the user include movement. In addition, the apnea stage means a sleep stage in which breathing activity does not occur during the physical activities of the user, and the snoring stage means a sleep stage in which snoring occurs during the physical activities of the user.

The higher the proportion of the deep sleep occupied in the sleep stage and the lower the proportion of the other stages, the higher the quality of the sleep. Therefore, when each sleep stage can be defined through the present invention, information on the sleep quality of the user can be acquired, and therefore, a prescription corresponding to the sleep quality can be provided for each user at a later time.

The sleep-stage definition unit (17) is formed to define a sleep state of each stage in the entire sleep stage using an analysis result of the Doppler signal analysis unit (15). Each stage in the entire sleep stage includes at least one among the sleep entry stage, deep sleep stage, tossing and turning stage, apnea stage, and snoring stage as described above. The sleep-stage definition unit (17) acquires an analysis result from the Doppler signal analysis unit (15) and determines which sleep stage the analysis result corresponds to using preset state determination criteria.

The preset state determination criteria may be a value set in advance using the ratio of the respiration spectrum energy and the heartbeat spectrum energy acquired by the Doppler signal analysis unit (15). As described above, since the magnitude of physical activities generating during the breathing motion is generally higher than the magnitude of physical activities generating during the heartbeat motion, the magnitude of the respiration spectrum energy appears to be higher than the magnitude of the heartbeat spectrum energy in a stable sleep stage. In addition, in the snoring stage, as high frequency vibration is captured, an additional spectrum other than the respiration spectrum energy and the heartbeat spectrum energy may appear. In addition, in the apnea stage, since a breathing motion does not occur or slightly occurs, the magnitude of the respiration spectrum energy may decrease as low as the magnitude of the heartbeat spectrum energy.

The sleep-stage definition unit (17) of an embodiment of the present invention may define a stage in which the spectrum ratio, which is the ratio of the respiration spectrum energy to the heartbeat spectrum energy, is 5:5 or higher as a deep sleep stage.

In addition, the sleep-stage definition unit (17) may define a stage in which the non-periodic spectrum energy is acquired to be lower than a preset magnitude, among the stages in which the spectrum ratio is lower than 5:5, as a snoring stage, and when the non-periodic spectrum energy is not acquired for a predetermined period of time, the stage may be defined as an apnea stage.

In another embodiment of the present invention, the sleep-stage definition unit (17) may define a sleep stage using the sleep entry stage, in addition to the spectrum ratio. Since it is general that the tossing and turning, apnea, or snoring does not occur in the sleep entry stage like in the deep sleep stage, the sleep-stage definition unit (17) of the present invention may be formed to acquire a sleep entry stage first and then determine a deep sleep stage based on the acquired sleep entry stage.

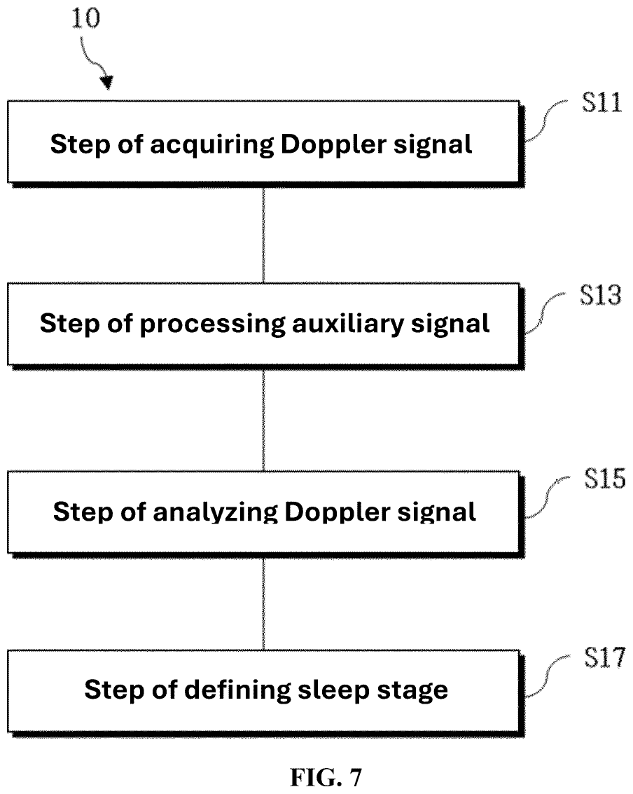

Meanwhile, FIG. 7 shows a flowchart illustrating a non-contact sleep monitoring method according to an embodiment of the present invention. Hereinafter, although it is described that a non-contact sleep monitoring method of the present invention is performed using the system of FIG. 6 for convenience of explanation, the present invention is not necessarily limited thereto.

A non-contact sleep monitoring method (10) according to an embodiment of the present invention may be formed to be installed in a space where a user lives and sleeps, such as a master bedroom or the like, to detect sleep when the user falls asleep, acquire Doppler signals for biological activities, and define a sleep state of the user by analyzing the acquired Doppler signals. To this end, the present invention may use a biological activity measurement device using a radar previously installed in the space described above. As shown in FIG. 7, the non-contact sleep monitoring method (10) according to an embodiment of the present invention may be formed to include a step of acquiring a Doppler signal (S11), a step of processing an auxiliary signal (S13), a step of analyzing the Doppler signal (S15), and a step of defining a sleep stage (S17).

The step of acquiring a Doppler signal (S11) is formed to acquire, by the Doppler signal acquisition unit (11), a Doppler signal including biological activity information from a radar installed in a user's sleeping space. The Doppler signal is formed to include a respiration Doppler signal, which is information on breathing of the user, and a heartbeat Doppler signal, which is information on heartbeat of the user. The Doppler signal may be defined as a signal that includes a Doppler frequency generated by movement of the user when the radar transmits radio waves to the user and the transmitted radio waves return. In an embodiment of the present invention, the respiration Doppler signal and the heartbeat Doppler signal may be Doppler signals with respect to the change in the movement of body organs that move while breathing and body organs that move while the heart is beating.

The step of processing an auxiliary signal (S13) is formed to acquire an auxiliary signal using an auxiliary biological activity information acquisition device further provided in the user's sleeping space, and acquire information on the movement (tossing and turning, or the like) of body parts other than breathing or heartbeat of the user by analyzing the acquired auxiliary signal. To this end, the step of processing an auxiliary signal (S13) may acquire measurement results from a thermal image sensor, which is an auxiliary biological activity information acquisition device. The thermal image sensor may be provided to measure the movement of the body of the user.

The step of processing an auxiliary signal (S13) may continuously acquire contour information of the user's body using the thermal image sensor. When contour information of the user's body is continuously acquired using the thermal image sensor, changes in the contour information generated due to the movement, such as tossing and turning or the like, of the user can be acquired.

The body contour information acquired at the step of processing an auxiliary signal (S13) may be used at the step of analyzing the Doppler signal (S15) described below, and as an example, when the step of analyzing the Doppler signal (S15) acquires non-periodic spectrum energy using information on the time of acquiring a tossing and turning motion or a snoring motion expressed as non-periodic spectrum energy, the non-periodic spectrum energy may be used as auxiliary information for determining what kind of biological activity information the energy means.

The step of analyzing the Doppler signal (S15) is formed to acquire spectrum energy at preset intervals by analyzing, by the Doppler signal analysis unit, the Doppler signals acquired through the step of acquiring a Doppler signal (S11). The step of analyzing the Doppler signal (S15) may acquire spectrum energy by applying a preset function to the Doppler signals, and acquire respiration spectrum energy and heartbeat spectrum energy, which are spectrum energies of the respiration Doppler signal and the heartbeat Doppler signal, by analyzing the acquired spectrum energy according to preset criteria.

In an embodiment of the present invention, the step of analyzing the Doppler signal (S15) may use a fast Fourier transform (FFT) as the preset function used to acquire spectrum energy from the Doppler signals at preset intervals. The Fourier transform is a well-known function and means a transformation that decomposes signals sampled in time or space into components of time frequency or space frequency. When the Fourier transform is used, the step of analyzing the Doppler signal (S15) may convert the Doppler signals acquired at preset intervals into spectrum energy of a specific frequency component.

The step of analyzing the Doppler signal (S15) according to an embodiment of the present invention is formed to confirm, when the spectrum energy of a specific frequency component is acquired, whether the acquired spectrum energy of a specific frequency component is spectrum energy generated periodically. The step of analyzing the Doppler signal (S15) may be formed to continuously acquire spectrum energy of the Doppler signals acquired at preset intervals and determine whether the acquired continuous spectrum energy has periodicity. Here, when the spectrum energy has periodicity, the spectrum energy may be determined as spectrum energy of breathing or heartbeat, and when the spectrum energy has no periodicity, the spectrum energy may not be determined as spectrum energy of breathing or heartbeat.

The step of analyzing the Doppler signal (S15) may confirm whether the non-periodic spectrum energy has an energy higher than a preset magnitude, and when it has an energy higher than a preset magnitude, the spectrum energy may be determined as spectrum energy of the Doppler frequency generated due to body movement of the user. In addition, it may be formed to determine, when the spectrum energy has an energy lower than a preset magnitude, the spectrum energy as spectrum energy of the Doppler frequency generated due to snoring of the user.

In addition, the step of analyzing the Doppler signal (S15) according to an embodiment of the present invention may perform analysis on periodic spectrum energy using a preset determination algorithm, and separately acquire respiration spectrum energy and heartbeat spectrum energy as a result of the analysis.

In addition, the step of analyzing the Doppler signal (S15) according to an embodiment of the present invention may identify the type of the non-periodic spectrum energy using the auxiliary signal acquired at the step of processing an auxiliary signal (S13) described above. Here, the step of analyzing the Doppler signal (S15) may be formed to acquire non-periodic spectrum energy of tossing and turning (body movement) of the user using, for example, thermal image information, and determine the acquired non-periodic spectrum energy as a noise signal and remove it from data for analyzing sleep stages described below.

In addition, in another embodiment, when an energy of a preset magnitude that can distinguish body movement from snoring using the non-periodic spectrum energy is not defined, the step of analyzing the Doppler signal (S15) may define an energy of a preset magnitude that can distinguish the two movements using the auxiliary signal acquired through a thermal image sensor.

The step of defining a sleep stage (S17) is formed to define, when the step of analyzing the Doppler signal (S15) according to an embodiment of the present invention continuously acquire spectrum energy of a specific frequency component, and determining whether or not the spectrum energy periodically occurs is completed, a sleep state in each stage of the entire sleep stage using an analysis result.

The entire sleep stage may include at least one among a sleep entry stage, a deep sleep stage, a tossing and turning stage, an apnea stage, and a snoring stage. The sleep entry stage means a stage in which the user enters sleep, and this is a stage in which whether the user begins to sleep can be determined, which occurs statistically in almost all users. In addition, the deep sleep stage means a sleep stage in which physical activities of the user are maintained stably, while the tossing and turning stage means a sleep stage in which the physical activities of the user include movement. In addition, the apnea stage means a sleep stage in which breathing activity does not occur during the physical activities of the user, and the snoring stage means a sleep stage in which snoring occurs during the physical activities of the user.

The higher the proportion of the deep sleep occupied in the sleep stage and the lower the proportion of the other stages, the higher the quality of the sleep. Therefore, when each sleep stage can be defined through the present invention, information on the sleep quality of the user can be acquired, and therefore, a prescription corresponding to the sleep quality can be provided for each user at a later time.

The step of defining a sleep stage (S17) is formed to define, by the sleep-stage definition unit, a sleep state of each stage in the entire sleep stage using an analysis result of the step of analyzing the Doppler signal (S15). Each stage in the entire sleep stage includes at least one among the sleep entry stage, deep sleep stage, tossing and turning stage, apnea stage, and snoring stage as described above. The step of defining a sleep stage (S17) acquires an analysis result from the step of analyzing the Doppler signal (S15) and determines which sleep stage the analysis result corresponds to using preset state determination criteria.

The preset state determination criteria may be a value set in advance using the ratio of the respiration spectrum energy and the heartbeat spectrum energy acquired at the step of analyzing the Doppler signal (S15). As described above, since the magnitude of physical activities generating during the breathing motion is generally higher than the magnitude of physical activities generating during the heartbeat motion, the magnitude of the respiration spectrum energy appears to be higher than the magnitude of the heartbeat spectrum energy in a stable sleep stage. In addition, in the snoring stage, as high frequency vibration is captured, an additional spectrum other than the respiration spectrum energy and the heartbeat spectrum energy may appear. In addition, in the apnea stage, since a breathing motion does not occur or slightly occurs, the magnitude of the respiration spectrum energy may decrease as low as the magnitude of the heartbeat spectrum energy.

The step of defining a sleep stage (S17) of an embodiment of the present invention may define a stage in which the spectrum ratio, which is the ratio of the respiration spectrum energy to the heartbeat spectrum energy, is 5:5 or higher as a deep sleep stage.

In addition, the step of defining a sleep stage (S17) may define a stage in which the non-periodic spectrum energy is acquired to be lower than a preset magnitude, among the stages in which the spectrum ratio is lower than 5:5, as a snoring stage, and when the non-periodic spectrum energy is not acquired for a predetermined period of time, the stage may be defined as an apnea stage.

In another embodiment of the present invention, the step of defining a sleep stage (S17) may define a sleep stage using the sleep entry stage, in addition to the spectrum ratio. Since it is general that the tossing and turning, apnea, or snoring does not occur in the sleep entry stage like in the deep sleep stage, the step of defining a sleep stage (S17) of the present invention may be formed to acquire a sleep entry stage first and then determine a deep sleep stage based on the acquired sleep entry stage.

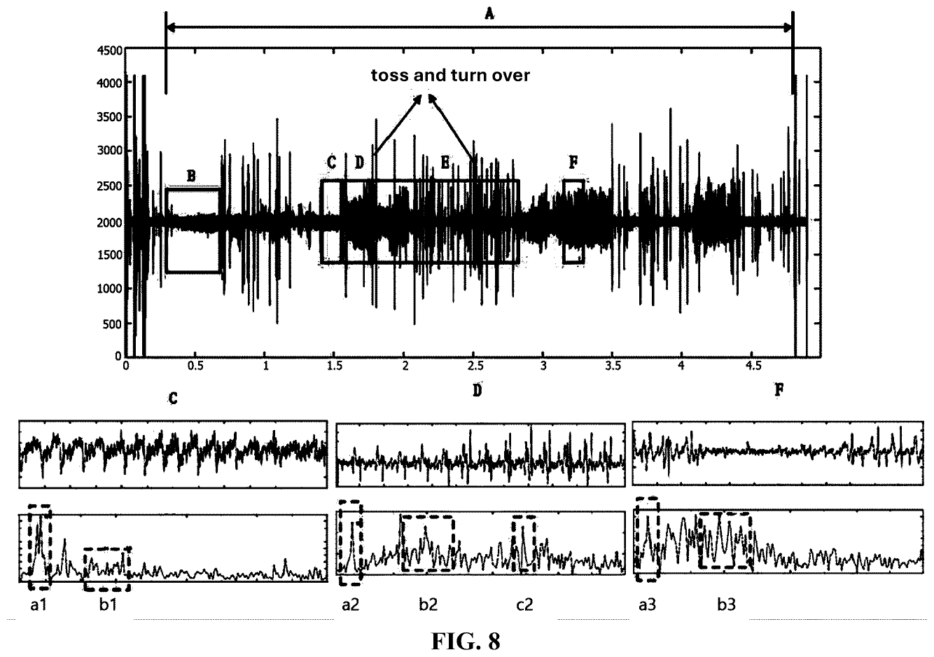

Meanwhile, FIG. 8 is a graph showing a result of an actual simulation using an embodiment of the present invention. Referring to FIG. 8, the entire sleep stage may be represented as A. Stage B is the sleep entry stage, which is a stage that occurs statistically when almost all users enter sleep as described above, and it may be a stage in which sleep of a user actually begins, which is a stage of 10 to 40 minutes, appearing as 20 to 30 minutes in average. Stage C is a deep sleep stage, which is a stable sleep stage, D is a snoring stage, E is a tossing and turning stage, and F is an apnea stage.

Stage C is a stable deep sleep stage, in which the magnitude of the respiration spectrum energy (a1) appears to be sufficiently higher than the magnitude of the heartbeat spectrum energy (b1). In addition, observing the periodicity of the spectrum energy, it can be confirmed that the spectrum energy does not include irregular peaks and repeats at a regular cycle. Therefore, in an embodiment of the present invention, this sleep stage may be defined as a deep sleep stage. At this point, in the present invention, as described above, a stage in which the ratio of respiration spectrum energy (a1) and heartbeat spectrum energy (b1) is 5:5 or higher may be defined as a deep sleep stage, and in another embodiment, a stage having a periodicity greater than or equal to a preset similarity and an energy lower than or equal to a preset magnitude compared to the sleep entry stage, i.e., stage B, may be defined as a deep sleep stage.

Stage D is a snoring stage, which is a stage in which although the magnitude of the respiration spectrum energy (a2) is formed to be higher than the magnitude of the heartbeat spectrum (b2), snoring spectrum energy (c2), which does not appear in the other stages, is additionally measured. In stage D, it can be confirmed that a spectrum energy like c2 is measured at a frequency higher than the frequency of breathing or heartbeat. Therefore, the sleep-stage definition unit and the step of defining a sleep stage of the present invention may define stage D as a snoring stage, in which the user is snoring.

Stage F is an apnea stage, in which the magnitude of the respiration spectrum energy (a3) appears to be similar to the magnitude of the heartbeat spectrum energy (b3). More specifically, this is a stage in which the ratio of the respiration spectrum energy (a3) and the heartbeat spectrum energy (b3) appears to be lower than 5:5, and the snoring spectrum energy (c2) is not measured, unlike stage D. This is since that as the breathing activity in the body of the user is weak or does not exist in the process of acquiring a Doppler signal using a radar, the magnitude of the acquired respiration spectrum energy (a3) is reduced greatly. Therefore, like the spectrum analysis result for stage F of FIG. 8, when the magnitude of the respiration spectrum energy (a3) is compared to the magnitude of the heartbeat spectrum energy (b3), and the ratio is lower than 5:5, the sleep-stage definition unit and the step of defining a sleep stage according to an embodiment of the present invention may define stage F as an apnea stage.

Stage E is a stage in which tossing and turning occurs, which is a stage in which spectrum energy having an energy of high magnitude compared to the spectrum energy detected in stages B, C, D, and Fis acquired. This is generation of a biological activity having a very high energy compared to the respiration spectrum energy or the heartbeat spectrum energy, and the present invention is formed to analyze such a biological activity as tossing and turning.

In summary, the system and method of FIGS. 6 and 7 are formed to acquire and analyze Doppler signals of biological activities of a user using a radar, and define a sleep state of each sleep stage using respiration spectrum energy and heartbeat spectrum energy of the Doppler signals, and results of experiments on the sleep states are shown in FIG. 8. As the present invention described in FIGS. 6 to 8 is used, there is an effect of confirming the sleep state of a user and responding to generation of an emergency situation or providing contents for improving sleep quality of the user using the confirmed sleep state.

Although an embodiment of the present invention has been described above, the spirit of the present invention is not limited to the embodiment presented in the present specification, and although those skilled in the art who understand the spirit of the present invention may easily suggest other embodiments by adding, changing, deleting, or supplementing components within the scope of the same spirit, it will be said that this is also within the scope of the present invention.

### INDUSTRIAL APPLICABILITY

The non-face-to-face non-contact fall monitoring system and the non-contact sleep monitoring system and method may increase reliability of fall detection while protecting privacy of a user, determine fall of the user in real time in a non-face-to-face non-contact method, rather than a wearable method, although the user does not wear a separate device, determine a risk level when it is determined as a fall and inform a guardian or medical staff of the risk to guide them to prevent or respond to the risk according to the fall in advance, protect privacy of the user by processing thermal images during fall monitoring and sleep monitoring to create a closed curve, a labeling image, and a spectrogram, analyze a risk level of the user and guide to properly respond thereto on the basis of biological signals of the user and image-processed thermal images, not disturb user's sleep as biological activity information is acquired using a radar while the user sleeps without affecting the user's body, and accurately determine a sleep state in each sleep stage of the user using the user's breathing and heartbeat information.

1. A non-face-to-face non-contact fall detection system comprising:
a radar sensor unit for transmitting a radar signal toward a user, receiving the radar signal reflected from the user, and generating a time series Doppler radar signal using a Doppler radar algorithm;
a thermal image sensor unit for generating thermal image data by photographing the user from a top of the user;
a radar signal pattern analysis unit for receiving the time series Doppler radar signal from the radar sensor unit, and analyzing a state of the user on the basis of the received time series Doppler radar signal;
a thermal image data processing unit for receiving the generated thermal image data from the thermal image sensor unit, and analyzing the state of the user by processing the received thermal image data; and
a fall determination unit for determining whether or not a fall has occurred on the basis of state data of the user received from the radar signal pattern analysis unit and the thermal image data processing unit.

2. The system according to claim 1, wherein the radar signal pattern analysis unit separates a movement change signal and a biological signal from the time series Doppler radar signal received from the radar sensor unit, and analyzes the state of the user using the separated movement change signal as a time series movement Doppler radar signal.

3. The system according to claim 2, wherein the radar signal pattern analysis unit generates a first state data indicating that a fall has occurred, when an amplitude size becomes 0 after a yin-yang change occurs in a data having an amplitude size of a predetermined level or higher in the time series movement Doppler radar signal within a first time, and generates a second state data indicating a state in which the user slowly falls down and a fall is suspected, when a yin-yang change occurs in the data having an amplitude size of a predetermined level or higher in the time series movement Doppler radar signal after the first time.

4. The system according to claim 3, wherein the thermal image data processing unit processes the thermal image data through the steps of:
determining an analysis range from the received thermal image data;
determining an edge of the user within the determined analysis range; and
acquiring a closed-curved image by processing the thermal image data to have a closed-curved shape from the determined edge using a preset threshold value.

5. The system according to claim 4, wherein the thermal image data processing unit forms a labeled image by further including the step of performing image labeling on the closed-curved image.

6. The system according to claim 5, wherein the thermal image data processing unit forms a spectrogram by further including the step of forming a spectrogram of the labeled image.

7. The system according to claim 3, wherein the thermal image data processing unit forms fourth to sixth state data by determining the state of the user as one among fourth to sixth states based on at least one among the closed-curved image, the labeled image, and the spectrogram, wherein the fourth state data indicates a state in which the user is positioned on an object at a predetermined height from a floor, the fifth state data indicates a state in which the user is falling from the object positioned at a predetermined height from the floor, and the sixth state data indicates a state in which the user has fallen from the object positioned at a predetermined height from the floor.

8. The system according to claim 7, wherein when the fall determination unit receives the first state data from the radar signal pattern analysis unit, and sequentially receives all the fourth state data, the fifth state data, and the sixth state data from the thermal image data processing unit, and the fourth state data is generated before a time point of generating the first state data, the fall determination unit determines that the user has fallen, and generates a first signal indicating that a fall of the user has occurred; when the fall determination unit receives the first state data determined as a first state from the radar signal pattern analysis unit, and sequentially receives all the sixth state data, the fifth state data, and the fourth state data from the thermal image data processing unit, and the sixth state data is generated before the time point of generating the first state data, the fall determination unit determines that the user has not fallen, and generates a second signal indicating that a fall has not occurred; when the fall determination unit receives the second state data indicating that a fall is suspected from the radar signal pattern analysis unit, and sequentially receives all the fourth state data, the fifth state data, and the sixth state data from the thermal image data processing unit, and the fourth state data is generated before the time point of generating the first state data, the fall determination unit determines that the user has fallen, and generates a third signal indicating that the user slowly falls down and a fall of the user has occurred; when the fall determination unit receives the third state data from the radar signal pattern analysis unit, and receives only the fourth state data from the thermal image data processing unit even after a fourth time from a time point of receiving the third state data, the fall determination unit generates a fourth signal indicating that the user is at a high risk of falling; and when the fall determination unit receives the first state data or the second state data from the radar signal pattern analysis unit, sequentially receives all the fourth state data, the fifth state data, and the sixth state data from the thermal image data processing unit, receives again the first state data or the second state data from the radar signal pattern analysis unit after the fourth time, and sequentially receives the sixth state data, the fifth state data, and the fourth state data from the thermal image data processing unit, the fall determination unit determines that the fallen user has returned to an original position, and generates a fifth signal indicating that the user has returned to the original position.

9. The system according to claim 8, wherein the radar signal pattern analysis unit analyzes the separated biological signal, and generates the sixth signal informing a dangerous situation when the biological signal is different from a biological signal pattern of a predetermined period as much as a predetermined range or more.

10. The non-contact fall and sleep monitoring system according to claim 9, further comprising a fall risk level determination unit, wherein when the fall risk level determination unit continuously receives the sixth signal from the radar signal pattern analysis unit during a fifth time, receives the first signal from the fall determination unit, and does not continuously receive the fourth signal within the fifth time, the fall risk level determination unit determines that the user is continuously in a fallen state and the biological signals are in a bad state, and generates a seventh signal informing that the user is in a very high risk due to the falling; when the fall risk level determination unit does not receive the sixth signal from the radar signal pattern analysis unit during the fifth time, and does not receive the fourth signal from the fall determination unit during the fifth time, the fall risk level determination unit generates an eighth signal indicating that the biological signals of the user are stable although the user is continuously in a fallen state; and when the fall risk level determination unit receives the third signal from the fall determination unit, the fall risk level determination unit generates a ninth signal informing that it is high probable that the user may fall.

11. The system according to claim 10, further comprising an alarm unit, wherein when the seventh signal is received from the fall risk level determination unit, the alarm unit generates a first alarm signal informing that the user is in a very critical state, and transmits the first alarm signal to both a predetermined guardian and medical staff; when the eighth signal is received from the fall risk level determination unit, the alarm unit generates a second alarm signal informing a fallen state of the user, and transmits the second alarm signal to a predetermined guardian or medical staff; and when the ninth signal is received from the fall risk level determination unit, the alarm unit generates a third alarm signal informing that falling of the user is imminent, and transmits the third alarm signal to a predesignated guardian or medical staff.

12. A non-contact sleep monitoring system comprising:
a Doppler signal acquisition unit for acquiring a Doppler signal including biological activity information using a radar;
an auxiliary signal processing unit for acquiring a tossing and turning motion as an auxiliary signal using a thermal image sensor;
a Doppler signal analysis unit for acquiring spectrum energy at preset intervals by analyzing the Doppler signal, determining whether the spectrum energy is acquired periodically, and performing, when the spectrum energy is determined as non-periodic spectrum energy, classification of the non-periodic spectrum energy using the auxiliary signal; and
a sleep-stage definition unit for defining a sleep state of each stage in an entire sleep stage using a combination of a ratio of respiration spectrum energy and heartbeat spectrum energy, among the spectrum energy, and the non-periodic spectrum energy.

13. The system according to claim 12, wherein the Doppler signal includes a respiration Doppler signal that acquires information on breathing of a user and a heartbeat Doppler signal that acquires information on heartbeat of the user.

14. The system according to claim 13, wherein the Doppler signal analysis unit acquires the spectrum energy by performing a fast Fourier transform on the Doppler signal.

15. The system according to claim 14, wherein the sleep-stage definition unit defines a stage in which the non-periodic spectrum energy does not exist, among stages where the ratio of respiration spectrum energy and heartbeat spectrum energy is 5:5 or higher in the entire sleep stage, as a deep sleep stage, and defines a stage in which the non-periodic spectrum energy does not exist, among stages where the ratio of respiration spectrum energy and heartbeat spectrum energy is lower than 5:5, as an apnea stage.

16. The system according to claim 15, wherein the sleep-stage definition unit defines a stage in which the non-periodic spectrum energy exists and the non-periodic spectrum energy appears to be higher than or equal to a preset magnitude as a tossing and turning stage, and defines a stage in which the non-periodic spectrum energy appears to be lower than or equal to a preset magnitude in the apnea stage as a snoring stage.

17. A non-contact sleep monitoring system comprising:
a Doppler signal acquisition unit for acquiring a Doppler signal including biological activity information using a radar;
an auxiliary signal processing unit for acquiring a tossing and turning motion as an auxiliary signal using a thermal image sensor;
a Doppler signal analysis unit for acquiring spectrum energy at preset intervals by analyzing the Doppler signal, determining whether the spectrum energy is acquired periodically, and performing, when the spectrum energy is determined as non-periodic spectrum energy, classification of the non-periodic spectrum energy using the auxiliary signal; and
a sleep-stage definition unit for defining sleep states of the other sleep stages using a preset ratio range and the non-periodic spectrum energy on the basis of an average of the spectrum energy of a sleep entry stage that satisfies preset criteria in an entire sleep stage.

18. A non-contact sleep monitoring method comprising:
a Doppler signal acquisition step of acquiring, by a Doppler signal acquisition unit, a Doppler signal including biological activity information using a radar;
an auxiliary signal processing step of acquiring, by an auxiliary signal processing unit, a tossing and turning motion as an auxiliary signal using a thermal image sensor;
a Doppler signal analysis step of acquiring, by a Doppler signal analysis unit, spectrum energy at preset intervals by analyzing the Doppler signal, determining whether the spectrum energy is acquired periodically, and performing, when the spectrum energy is determined as non-periodic spectrum energy, classification of the non-periodic spectrum energy using the auxiliary signal; and
a sleep-stage definition step of defining, by a sleep-stage definition unit, a sleep state of each stage in an entire sleep stage using a combination of a ratio of respiration spectrum energy and heartbeat spectrum energy, among the spectrum energy, and the non-periodic spectrum energy.

19. The method according to claim 18, wherein the Doppler signal includes a respiration Doppler signal that acquires information on breathing of a user and a heartbeat Doppler signal that acquires information on heartbeat of the user.

20. The method according to claim 19, wherein the Doppler signal analysis step acquires the spectrum energy by performing a fast Fourier transform on the Doppler signal.

21. The method according to claim 20, wherein the sleep-stage definition step defines a stage in which the non-periodic spectrum energy does not exist, among stages where the ratio of respiration spectrum energy and heartbeat spectrum energy is 5:5 or higher in the entire sleep stage, as a deep sleep stage, and defines a stage in which the non-periodic spectrum energy does not exist, among stages where the ratio of respiration spectrum energy and heartbeat spectrum energy is lower than 5:5, as an apnea stage.

22. The method according to claim 21, wherein the sleep-stage definition step defines a stage in which the non-periodic spectrum energy exists and the non-periodic spectrum energy appears to be higher than or equal to a preset magnitude as a tossing and turning stage, and defines a stage in which the non-periodic spectrum energy appears to be lower than or equal to a preset magnitude in the apnea stage as a snoring stage.

23. A non-contact sleep monitoring method comprising the steps of:
acquiring, by a Doppler signal acquisition unit, a Doppler signal including biological activity information using a radar;
acquiring, by an auxiliary signal processing unit, a tossing and turning motion as an auxiliary signal using a thermal image sensor;
acquiring, by a Doppler signal analysis unit, spectrum energy at preset intervals by analyzing the Doppler signal, determining whether the spectrum energy is acquired periodically, and performing classification of the spectrum energy using the auxiliary signal; and
defining, by a sleep-stage definition unit, sleep states of the other sleep stages using a preset ratio range and the non-periodic spectrum energy on the basis of an average of the spectrum energy of a sleep entry stage that satisfies preset criteria in an entire sleep stage.
